(19) United States
(12) Patent Application Publication
(10) Pub. No.: US 2023/0296625 A1
(43) Pub. Date: Sep. 21, 2023

(54) CXCL14 AS PREDICTIVE BIOMARKER FOR ACUTE LIVER FAILURE AND DEATH

(71) Applicant: University of Kansas, Lawrence, KS (US)

(72) Inventors: Hartmut Jaeschke, Overland Park, KS (US); Anup Ramachandran, Overland Park, KS (US); Nga T. Nguyen, Kansas City, MO (US)

(21) Appl. No.: 18/185,164

(22) Filed: Mar. 16, 2023

Related U.S. Application Data
(60) Provisional application No. 63/321,009, filed on Mar. 17, 2022.

(51) Int. Cl. G01N 33/68

(52) U.S. Cl. CPC G01N 33/6893; G01N 2333/521; G01N 2800/085; G01N 2800/52

(57) ABSTRACT

A method of determining a likelihood of liver transplant can be performed on a subject that has consumed acetaminophen at an initial time point and has been diagnosed with hepatotoxicity. The amount of CXCL14 in the blood samples to the series of time points can be measured to determine a trend in the amount of the CXCL14 in the blood samples over a time period beginning at the initial time point and continuing through the series of time points. The trend can be: (a) the amount of CXCL14 increases over the time period - the treatment is determined to be a liver transplant; (b) the amount of CXCL14 increases and then becomes substantially constant the treatment is determined to be a liver transplant; or (c) the amount of CXCL14 has a maximum (> 3000) and then decreases - the treatment is determined to exclude a liver transplant.

### CROSS-REFERENCE TO RELATED APPLICATIONS

This patent application claims priority to U.S. Provisional Application No. 63/321,009 filed Mar. 17, 2022, which provisional is incorporated herein by specific reference in its entirety.

### U.S. GOVERNMENT RIGHTS

This invention was made with government support under DK125465 awarded by the National Institutes of Health. The government has certain rights in the invention.

### BACKGROUND

### Field

The present disclosure relates to methods for predicting survival after acetaminophen-induced hepatotoxicity.

### Description of Related Art

Due to being identified for numerous indications, acetaminophen (N-acetyl-p-aminophenol, APAP) is commonly used as an over-the-counter medication. However, acetaminophen is the most common cause of acute liver failure (ALF) in the Western countries {Bernal, 2015}. The Rumack-Matthew monogram is standard practice for determining whether patients can be successfully treated with N-acetylcysteine (NAC). However, the Rumack-Matthew monogram is deficient and not useful in many cases. For example, if the patient has multiple APAP ingestions or presents more than 24 hours after ingestion. Moreover, treatment with NAC is often not effective in late-presenting patients and patients that have an acetaminophen level outside the treatment line of thereof indicating a very severe overdose.

Patients are often admitted to the hospital with no detectable level of acetaminophen, which causes difficulty for clinicians to diagnose patients as an APAP overdose and treat them accordingly. In addition, delayed NAC treatment can be detrimental to patient’s recovery due to impairment of regeneration {Akakpo, 2021}. Mild and moderate acetaminophen intoxication can be treated with supportive care at the hospital. However, severe acetaminophen overdose cases with high risk of developing acute liver failure may require liver transplantation. As such, it is critical for a clinician to have access to tools that allow them to accurately predict the prognosis of patients with an acetaminophen overdose for better management {Bernal, 1998}. Standard clinical assays like aminotransferases (ALT and AST), bilirubin and prothrombin are used to assess liver hepatotoxicity and liver function, but do not provide specific prognosis regarding a need of a liver transplant.

Patients who are above the treatment line on the Rumack-Matthew monogram are administered NAC, which is currently the only FDA approved antidote for acetaminophen-induced liver injury. NAC has a short therapeutic window and needs to be administered within 8-10 hours after acetaminophen ingestion for optimal efficacy; however, a significant number of patients only go to the hospital when they are beyond this time window. Hence, NAC is not very effective in these patients, and they can develop acute liver failure.

Given time and supportive care, inherent liver regenerative capacity in some patients with liver failure can repopulate areas of liver necrosis and they can recover liver function. However, in others this regenerative response is diminished, and they then progress to liver failure and encephalopathy, at which point a liver transplant is the only treatment available. The dilemma facing physicians is deciding who needs a liver transplant and who is likely to recover with supporting care at the earliest time possible. While circulating liver enzymes, such as ALT and AST, provide indication of liver injury and scoring systems (e.g.,. MELD and Child-Pugh Scores) provide information on severity of liver disease, there are currently no specific biomarkers that can determine which patients will inherently recover liver function and which patients are likely to deteriorate further and need a liver transplant. This decision is critical, since transplanting a liver into a patient who would have inherently recovered, subjects them to an unnecessary life-long regimen of immunosuppressants as well as the stress of major surgery and costs involved. On the other hand, not promptly transplanting a patient who is unlikely to recover by themselves, significantly increases the possibility of morbidity and mortality.

Thus, there is a need for a protocol to predict the opportunity of a person to survive acetaminophen-induced hepatotoxicity without a liver transplant.

### SUMMARY

In some embodiments, a method of determining a likelihood of liver transplant in a subject is provided. The method can be performed on a subject that has consumed acetaminophen at an initial time point and has been diagnosed with hepatotoxicity. The method can include obtaining a plurality of blood samples from the subject at a series of time points over a plurality of days and measuring an amount of CXCL14 in the blood sample at the series of time points. Then, the amount of CXCL14 in the blood samples can be compared to the series of time points to determine a trend in the amount of the CXCL14 in the blood samples over a time period beginning at the initial time point (or at a time of peak ALT) and continuing through the series of time points. The method can then include identifying a trend of: (a) the amount of CXCL14 increases over the time period; (b) the amount of CXCL14 increases and then becomes relatively constant; or (c) the amount of CXCL14 has a maximum and then decreases. The method can then include selecting the trend in the amount of CXCL14 in the blood over the time period that matches the trend of (a), (b), or (c). The treatment of the subject can be based on the trend in the amount of CXCL14 in the blood samples over the time period. The treatment can be selected from: when the amount of CXCL14 increases over the time period, the treatment is determined to be a liver transplant; when the amount of CXCL14 increases and then becomes substantially constant or relatively constant (e.g., within 1%, 2%, 5%, 10%), the treatment is determined to be a liver transplant; or when the amount of CXCL14 increases to a maximum and then decreases, the treatment is determined to exclude a liver transplant. A report can be prepared that identifies the treatment for the subject, and the report can be provided to the subject, or a medical professional, or a medical clinic, or a hospital or other treatment facility. In some aspects, the time period is at least 2 days. In some aspects, the time period is at least 3 days. In some aspects, in trend (c) the amount of CXCL14 starts at a maximum and then decreases. In some aspects, in trend (c) the amount of CXCL14 increases to a maximum and then decreases. When the trend (c) has a maximum below 3,000 pg/mL, the treatment is to forgo liver transplant.

In some aspects, when the amount of CXCL14 is at least 3,000 pg/mL, or at least 5,000 pg/mL in plasma from the blood, the treatment is the liver transplant. In some aspects, when the amount of CXCL14 is at least 6,000 pg/mL in plasma, the treatment is the liver transplant. Here, the amounts can be compared to a mouse model having been administered 300 mg/kg or 600 mg/kg acetaminophen. As such, the values in humans can be correlated to the 5,000 pg/mL and 10,000 pg/mL in mice.

In some embodiments, the methods can include measuring at least one of ALT, bilirubin, or prothrombin. The data from the measuring of at least one of ALT, bilirubin, or prothrombin can be used to support the identified treatment. For example, the measurement of ALT or prothrombin at a plurality of time points to observe the peak amount/concentration can be used to support the treatment. In some aspects, the time period is determined to start at the time of the peak ALT. In some aspects, the prothrombin peak is identified at day 1 after the initial time point (e.g., either consumption or ALT peak) along with a CXCL14 of at least 3,000 pg/mL, 5,000 pg/mL, or 6,000 pg/mL. Therefore, the determination can be made on day 1 after the initial time point.

In some embodiments, a method of treating a subject having hepatotoxicity is provided. The method can be performed on a subject having consumed acetaminophen at an initial time point and having been diagnosed with hepatotoxicity. A plurality of blood samples from the subject at a series of time points over a plurality of days, and an amount of CXCL14 in the blood sample can be measured at the series of time points. The method can include comparing the amount of CXCL14 in the blood samples to the series of time points to determine a trend in the amount of the CXCL14 in the blood samples over a time period beginning at the initial time point and continuing through the series of time points. The data trend (e.g., trend of the data points over the time period) can be identified as: (a) the amount of CXCL14 increases over the time period; (b) the amount of CXCL14 increases and then becomes substantially constant or relatively constant; or (c) the amount of CXCL14 has a maximum and then decreases. The method can include selecting the trend in the amount of CXCL14 in the blood over the time period that matches the trend of (a), (b), or (c). A treatment of the subject can be determined based on the trend in the amount of CXCL14 in the blood samples over the time period, wherein the treatment is selected from: when the amount of CXCL14 increases over the time period, the treatment is determined to be a liver transplant; when the amount of CXCL14 increases and then becomes substantially constant, the treatment is determined to be a liver transplant; or when the amount of CXCL14 increases to a maximum and then decreases, the treatment is determined to exclude a liver transplant. Once the treatment is identified, the method can include an entity coordinating performance of the treatment on the subject. Then, the subject can be treated as determined and reported. In some aspects, the initial time point can be the time point at which the ALT amount/concentration peaks.

In some embodiments, the method can include performing the treatment on the subject. In some aspects, the treatment includes the liver transplant. In some aspects, the treatment excludes the liver transplant.

In some aspects of the invention, the time period is at least 2 days. In other aspects of the invention, the time period is at least 3 days. In some aspects, in trend (c) the amount of CXCL14 starts at a maximum and then decreases. In some aspects, in trend (c) the amount of CXCL14 increases to a maximum and then decreases.

In some embodiments, when the amount of CXCL14 is at least 5,000 pg/mL in plasma from the blood, the treatment is the liver transplant. In some aspects, when the amount of CXCL14 is at least 10,000 pg/mL in plasma, the treatment is the liver transplant. In some aspects, the amounts of CXCL14 can be compared to a mouse model having been administered 300 mg/kg or 600 mg/kg acetaminophen. Accordingly, the amount of CXCL14 can be correlated to at least 5,000 pg/mL or at least 10,000 pg/mL in the model mice having the acetaminophen treatment.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A includes a graph showing plasma ALT (U/L) versus time for treatment of mice with acetaminophen at 300 mg/kg or 600 mg/kg.

FIG. 1B includes a graph showing plasma CXCL14 (pg/mL) versus time for treatment of mice with acetaminophen at 300 mg/kg or 600 mg/kg.

FIG. 1C includes a graph showing the CXCL14 mRNA versus time for treatment of mice with acetaminophen at 300 mg/kg or 600 mg/kg.

FIG. 2A includes a graph showing plasma ALT (U/L) versus time for mice that are survivors with no injury, mice that are survivors with high injury, or non-survivors.

FIG. 2B includes a graph showing plasma CXCL14 (pg/mL) versus time for treatment of mice that are survivors with no injury, mice that are survivors with high injury, or non-survivors.

FIG. 2C includes a graph showing the sensitivity versus the specificity for days 1, 2, and 3 as well as the AUC thereof.

FIG. 3A includes an image of a Western blot showing the APAP treatment has higher CXCL14 expression compared to healthy subjects.

FIG. 3B shows the different histograms for H&E for healthy versus APAP treatment.

FIG. 3C shows the different histograms for CXCL14 for healthy versus APAP treatment.

FIG. 3D shows the different histograms for CD68 for healthy versus APAP treatment.

FIG. 4A includes a graph that shows the plasma ALT (U/L) for surviving mice versus day after peak ALT.

FIG. 4B includes a graph that shows the plasma ALT (U/L) for non-surviving mice versus day after peak ALT.

FIG. 4C includes a graph that shows the bilirubin (mg/mL) for surviving mice versus day after peak ALT.

FIG. 4D includes a graph that shows the bilirubin (mg/mL) for non-surviving mice versus day after peak ALT.

FIG. 4E includes a graph that shows the plasma CXCL14 (pg/mL) for surviving mice versus day after peak ALT.

FIG. 4F includes a graph that shows the plasma CXCL14 (pg/mL) for non-surviving mice versus day after peak ALT.

FIG. 5A includes a graph that shows a correlation between ALT and CXCL14.

FIG. 5B includes a graph that shows a correlation between CXCL14 and bilirubin.

FIG. 6A includes a graph that shows the time for peak prothrombin times for survivors versus non-survivors.

FIG. 6B includes a graph that shows the sensitivity versus the specificity for peak prothrombin times as well as the AUC thereof.

FIG. 6C includes a graph that shows a correlation between CXCL14 and peak prothrombin times.

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology relates to using CXCL14 as a predictive biomarker for identifying the potential of survival after acetaminophen-induced hepatotoxicity and acute liver failure in humans. The CXCL14 can be measured from a biological sample (e.g., blood, plasma, liver biopsy, etc.) to determine whether patients are likely to (1) recover or (2) deteriorate after acute liver injury due to an acetaminophen (APAP) exposure or overdoes. The data provided herein shows that CXCL14 is a cytokine secreted predominantly by surviving hepatocytes around areas of necrosis as a marker, which can differentiate between patients who can inherently recover and survive from those needing a liver transplant for survival. As indicated in the data, CXCL14 clearly delineates these two groups of patients without overlap. Thus, CXCL14 can be used to determine which of the two groups the patient is likely to fall into, which allows for determining the appropriate treatment to maximize the potential of survival. CXCL14 is a well-known defined protein, which is chemokine (C-X-C motif) ligand 14 (CXCL14) is a small cytokine belonging to the CXC chemokine family that is also known as BRAK (for breast and kidney-expressed chemokine).

Chemokine (C-X-C motif) Ligand 14 Identifiers: Symbol - CXCL14; Alt. symbols - SCYB14, BRAK, NJAC, bolekine, Kec, MIP-2g, BMAC, KS1; NCBI gene - 9547; HGNC - 10640; OMIM - 604186; PDB- 2HDL; RefSeq - NM_004887; UniProt - O95715; Other data - Locus Chr. 5 q31.

In some embodiments, the CXCL14 can be used as a biomarker to distinguish acute liver failure patients who can inherently repair the liver damage, recover liver function and survive from patients with compromised liver regeneration who would need a liver transplant for survival. Accordingly, the CXCL14 can be used as a biomarker to identify patients that are unlikely to repair the liver damage and recover the liver function, who can then be treated by receiving a liver transplant. Correspondingly, the CXCL14 can be used as a biomarker to identify patients that are likely to recover on their own without a liver transplant. Accordingly, the use of CXCL14 as a biomarker can identify patients that can recover and thereby are spared the stress and cost of major surgery as well as life-long immunosuppression.

In some embodiments, a method of identifying a treatment for a patient having acetaminophen exposure is provided. The method can include providing a patient that has been exposed to acetaminophen and then taking a biological sample from the patient. The biological sample is then assessed for the CXCL14. In some instances, the CXCL14 will provide an indication that the patient recovers, and thereby the identified treatment is to forego a liver transplant. In other instances, the CXCL14 will provide an indication that the patient will not recover, and thereby the identified treatment is to undergo a liver transplant. Such liver transplant can be at least a partial liver transplant to transplanting a full liver.

In some embodiments, the methods can include measuring CXCL14 by any means, such as ELISA, in patients with acute liver failure. That is, the provided patient is already known as having liver toxicity (hepatotoxicity) or acute liver failure. The data of the CXCL14 can provide prognostic information to clinicians regarding the recovery potential of the liver, which is used to determine whether or not the patient should undergo a liver transplant. Thus, the CXCL14 data can provide rationale guidance to a medical practitioner to make decisions of which patients to be put on a waitlist for liver transplantation and which patients to omit from a liver transplantation procedure. Therefore, a step of any of the methods can include putting the patient on such a waitlist for liver transplantation as well as performing any tests in order to determine the suitable donor of the liver. The methods may also include matching a donor liver to the subject.

In some embodiments, the measurement of the CXCL14 biomarker in human patients with acute liver failure can be performed as descried herein. The conceptual link to the human patients has been established based on outcomes of the mouse model of acetaminophen-induced liver injury that is described herein. The mouse model has been shown to replicate the human pathophysiology and that the regenerative response of the liver differs depending on the dose of acetaminophen used in the model.

In some embodiments, a method is provided for determining that a subject has acetaminophen toxicity. The method can include providing a subject suspected of having acetaminophen toxicity, and obtaining a biological sample from the subject. The CXCL14 can be measured as a biomarker in the biological sample. The amount of CXCL14 in the biological sample can be determined at one or more time points. The amount of CXCL14 or time curve thereof can be compared to a defined level or defined time curve of CXCL14. For example, the defined level or defined time curve of CXCL14 is based on one of a presence of liver toxicity or lack of liver toxicity. For example, the amount of CXCL14 for lack of liver toxicity will be low or have a flat curve. On the other hand, the amount of CXCL14 showing liver toxicity will be high or have a steep curve, where the curve may not flatten or have a downward arc. Flatting without a downward arc may also indicate the liver toxicity that needs a liver transplant. Also, the patient’s CXCL14 curve can be compared against both of these two curves, which can serve as positive (needing liver transplant) or negative (not needing liver transplant) controls.

In some embodiments, the defined level of CXCL14 or defined time curve of CXCL14 is obtained based on a level of CXCL14 or a time curve of CXCL14 in a plurality of subjects with acetaminophen toxicity. When the amount of CXCL14 or time curve thereof matches the defined level of CXCL14 or defined time curve of CXCL14 for indications of acetaminophen toxicity, the subject likely has acetaminophen toxicity. On the other hand, if the CXCL14 or time curve thereof matches the levels or time curves indicative of no acetaminophen toxicity, then the subject likely does not have acetaminophen toxicity. Here, acetaminophen toxicity can indicate need for liver transplant based on the presence of liver toxicity.

In some embodiments, the defined level of CXCL14 or defined time curve of CXCL14 is obtained based on a level of CXCL14 or a time curve of CXCL14 in a plurality of subjects without acetaminophen toxicity. In this example, the defined level is an average or mean of CXCL14 values of the plurality of subjects without acetaminophen toxicity. Also, the time curve can be based on CXCL14 time curves of the plurality of subjects without acetaminophen toxicity. This can be used as a negative comparison without the need for a transplant.

In some embodiments, the assessment of acetaminophen toxicity can include measuring a second biomarker in the biological sample. Then, the amount of the second biomarker in the biological sample can be determined at one or more time points. The amount of the second biomarker or time curve thereof can be compared to a defined level or defined time curve of the second biomarker. In some aspects, the defined level or defined time curve of the second biomarker can be obtained based on a level or time curve of the second biomarker in at least one subject without acetaminophen toxicity. In other aspects, the defined level or defined time curve of the second biomarker is obtained based on a level or time curve of the second biomarker in at least one subject with acetaminophen toxicity. Therefore, either having toxicity (needing transplant) or not having toxicity (no transplant), can be the basis for comparison. Also, the comparison can be based on a trend, with a steep slope indicating liver toxicity whereas a gradual slope with a plateau and downward trend indicates no liver toxicity.

In some embodiments, the method can include determining that the amount of CXCL14 is higher than the defined level of CXCL14 or the time curve of CXCL14 does not decrease after an initial time curve maximum. If this occurs, then it is determined that the subject has acetaminophen toxicity. When the subject has acetaminophen toxicity, the method can include generating a report on the acetaminophen toxicity and expected health status of a failing liver and need for transplant in view of the acetaminophen toxicity. The report can be provided to the subject for consideration, whether in verbal or written or electronic form. The report can also include the recommendation for a liver transplant, and optionally liver transplant providers. Also, the report can include information about a waitlist for a donated liver. The patient can then be put on the waitlist, and can undergo further examinations for matching with a donor liver.

In some embodiments, the methods can include identifying the acetaminophen toxicity causing liver failure in the subject, and then recommending that the patent receive at least a partial liver transplant. Accordingly, the methods can then include performing at least a partial liver transplant in the subject. The performance of the liver transplant can be by a medical entity for the patient.

In some embodiments, the methods can include determining that the amount of CXCL14 is about the same as the defined level of CXCL14 that indicates no liver toxicity or that the liver will heal. In some aspects, the determination can be that the time curve of CXCL14 does decrease after an initial time curve low, slow increase (e.g., substantially negligible) and maximum. The analysis of the CXCL14 data can be used to determine that the subject has sufficient liver functionality and/or live liver cells. As a result, a report can be generated based on the sufficient liver functionality and/or live liver cells and expected health status of a healing liver, and the report can be provided to the subject. The report can indicate that no liver transplant is needed. The report can also include recommendations to improve the healing liver and any other associated treatments to regenerate the liver.

In some aspects, the CXCL14 data has a low slope to a maximum and/or downward arc, which indicates the liver may recover. This allows the medical professional to identify the subject as having a liver capable of healing. The medical professional can then treat the subject to improve health of the liver so as to promote liver healing.

In some embodiments, a method for determining a treatment protocol for a subject suspected of having acetaminophen toxicity can be performed. Such a method can include providing a subject suspected of having acetaminophen toxicity. For example, the subject may have already been diagnosed as having acetaminophen toxicity from over consumption or overdose. The method can include obtaining a biological sample from the subject and measuring CXCL14 as a biomarker in the biological sample, and determining an amount of CXCL14 in the biological sample at one or more time points. The amount of CXCL14 or time curve thereof can be compared to a defined level or defined time curve of CXCL14, wherein the defined level or defined time curve of CXCL14 is based on one of (1) a presence of higher liver toxicity and needing a liver transplant or (2) lower liver toxicity and not needing a liver transplant. Thus, either the curves indicating liver toxicity or the curves indicating no liver toxicity can be the basis of the comparison, where when the new data curve for the patient is obtained, it can be compared to either or both standard curves. This allows determination of whether the patient has liver toxicity and needs a transplant, or not.

In some embodiments, the defined level of CXCL14 or defined time curve of CXCL14 is obtained based on a level of CXCL14 or a time curve of CXCL14 in a plurality of subjects with acetaminophen toxicity needing a liver transplant. When the amount of CXCL14 or time curve thereof matches this defined level of CXCL14 or defined time curve of CXCL14, the subject needs a liver transplant.

In some embodiments, the defined level of CXCL14 or defined time curve of CXCL14 is obtained based on a level of CXCL14 or a time curve of CXCL14 in a plurality of subjects with lower acetaminophen toxicity or without a need for a liver transplant. In some aspects, the defined level is an average or mean of CXCL14 values of the plurality of subjects without acetaminophen toxicity or without a need for a liver transplant, or the time curve is based on CXCL14 time curves of the plurality of subjects without acetaminophen toxicity or without a need for a liver transplant.

In some embodiments, the methods can include measuring a second biomarker in the biological sample. This allows for determining an amount of the second biomarker in the biological sample at one or more time points, and comparing the amount of the second biomarker or time curve thereof to a defined level or defined time curve of the second biomarker. In some aspects, the defined level or defined time curve of the second biomarker is obtained based on a level or time curve of the second biomarker in at least one subject without acetaminophen toxicity or needing a liver transplant. In other aspects, the defined level or defined time curve of the second biomarker is obtained based on a level or time curve of the second biomarker in at least one subject with acetaminophen toxicity and needing a liver transplant.

In some embodiments, the methods can include determining that the amount of CXCL14 is higher than the defined level of CXCL14 (e.g., 3000-5000 pg/ml in humans) or the time curve of CXCL14 does not decrease after an initial time curve maximum. Then, it can be determined that the subject has acetaminophen toxicity and a need for a liver transplant. A report can be generated on the acetaminophen toxicity and expected health status of a failing liver in view of the acetaminophen toxicity and need for a liver transplant. Then, the subject can be provided with the report. In some aspects, the method can include identifying the acetaminophen toxicity causing liver failure in the subject; and performing at least a partial liver transplant in the subject. In other instances, when there is no liver toxicity causing liver failure and the liver is expected to survive without a transplant, the report can include instructions to forgo any liver transplant.

In some embodiments, the methods can include determining that the amount of CXCL14 is about the same as the defined level of CXCL14 or the time curve of CXCL14 of no liver toxicity or the liver being able to rejuvenate, or the curve does decrease after an initial time curve maximum. When these results occur, it can be determined that the subject has sufficient liver functionality and/or live liver cells. This allows for the generation of a report on the sufficient liver functionality and/or live liver cells and expected health status of a healing liver, which report can be provided to the subject.

In some embodiments, the method can include identifying the subject to have a liver capable of healing, and treating the subject to improve health of the liver so as to promote liver healing.

In some embodiments, plasma can be tested for CXCL14 levels in acute liver injury patients due to acetaminophen overdose, where the results can vary from mild to severe to lethal to demonstrate the difference in CXCL14 level. It was found that CXCL14 is increased in patients with high injury; however, its level is significantly elevated in the non-survivors after several days. In the survivors, CXCL14 is increased at the early stage but decreased as the patients progressed toward day 3. In contrast, the non-survivors have significantly higher CXCL14 at day 1 and the level was consistently elevated. The data suggest that high CXCL14 (without substantially decreasing) is a death predictor, which can be used in the clinic to shape clinician’s treatment plans and liver transplantation priority.

### EXAMPLES

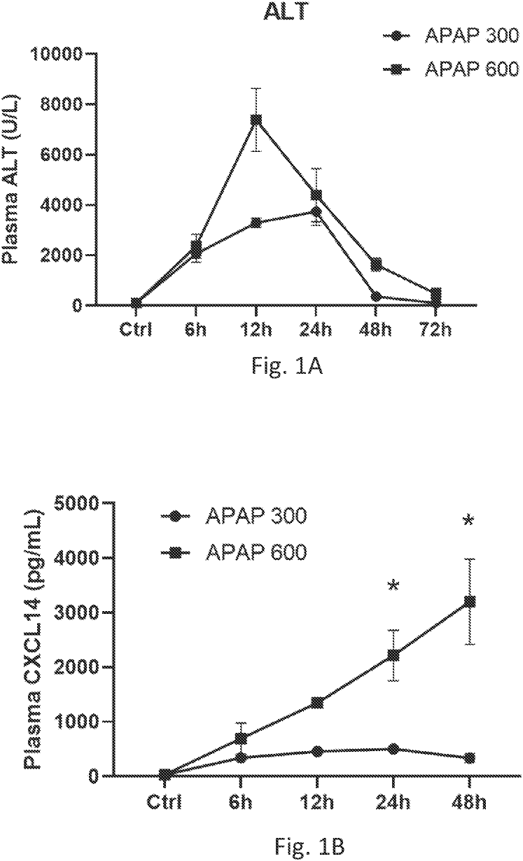

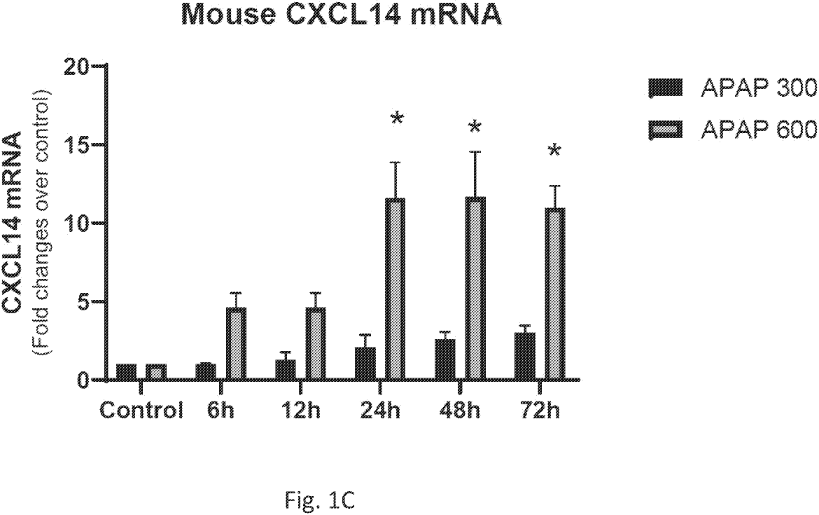

Mice treated with a moderate acetaminophen overdose of 300 mg/kg typically recover after liver injury by activating inherent regenerative mechanisms, while those administered with a severe overdose of 600 mg/kg typically do not recover and have significant mortality in this model. The temporal course of liver injury in this model was initially examined and it is evident that while ALT levels peak much higher in the mice with severe overdose (600 mg/kg) when compared to the 300 mg/kg dose (see FIG. 1A) they return to baseline in both doses by 72 hours. In contrast, plasma CXCL14 levels rapidly rise to very high levels in mice with the 600 mg/kg dose who do not recover when compared to the mice with the lower dose that do recover. As such, high levels of measured CXCL14 can indicate patients that need a liver transplant and low levels of CXCL14 can indicate patients that should forgo the liver transplant.

The elevated CXCL14 levels are shown to continue to rise and do not return to baseline as seen in mice with the 300 mg/kg dose (FIG. 1B). As such, the totality of the curve of the CXCL14 levels over time increasing without decreasing can indicate patients that need a liver transplant. On the other hand, CXCL14 levels that do not substantially increase over time or slightly increase then decrease can indicate patients that do not need a liver transplant. The distinction shown in FIG. 1B can provide for visual identification of the data where the steep slope in plasma CXCL14 indicates the need for a liver transplant, and the absence of a steep slope (or presence of a downward arc) indicates no need for a liver transplant. The slope can be based on scaling 1000 pg/mL evenly with a 6 hour time interval as shown in FIG. 1B.

To confirm that the elevated CXCL14 was being produced in the liver, mRNA levels were then examined in liver tissue from both groups and it is clearly evident that mice with a severe acetaminophen overdose significantly induce expression of CXCL14, which is sustained for long periods of time (FIG. 1C), even when the ALT levels have come back to baseline (FIG. 1A). Accordingly, either the CXCL14 protein (FIG. 1B) or the CXCL14 mRNA levels can be used for making the treatment determinations.

For FIGS. 1A-1C, the data shows the temporal course of the ALT or CXCL14 of fasted male C57BL/6J mice treated with the identified amount of acetaminophen. The liver and plasma samples were collected between 6-72 hours after acetaminophen exposure. FIG. 1A includes a graph that shows the ALT levels in plasma. FIG. 1B includes a graph that shows the CXCL14 levels in plasma as measured by ELISA. FIG. 1C shows the CXCL14 mRNA levels from liver samples of the acetaminophen treated mice.

A series of experiments were performed in order to evaluate the relevance of the findings from the mouse model (FIGS. 1A-1C), and whether the data can be relevant to humans. The data and characteristics of patient samples used for the analysis are depicted in Table 1. Plasma samples (N=60) were from acetaminophen overdose patients, where the plasma samples include 10 survivors with low injury, 30 survivors with high injury and 19 non-survivors. 10 healthy volunteer plasma samples were also obtained for comparison. Patients were categorized based on alanine amino transferase (ALT) level, with survivors with very mild ALT elevations at < 100 U/L categorized as low injury and survivors with peak ALT of 6200 U/L categorized as high injury. Non-survivors had a peak ALT similar to that of high injury survivors at 6000 U/L.

TABLE 1

| | Age | Female | Male | Peak bilirubin (mg/dL) | Peak Creatinine (mg/dL) | Admission APAP (mg/L) | Peak ALT | Peak AST |
|---|---|---|---|---|---|---|---|---|
| Survivors-No injury | 19-57 | 5 | 2 | 0.2-1.9 | 0.6-1.2 | 11-385 | 28-53 | 15-31 |
| Survivors-High | 19-76 | 14 | 16 | 1-15.9 | 0.8-10.2 | 0-616 | 943-13207 | 560-21700 |
| Non-survivors | 22-59 | 13 | 4 | 3.2-24.6 | 0.4-5.8 | 0-720 | 1113-13690 | 3751-22484 |

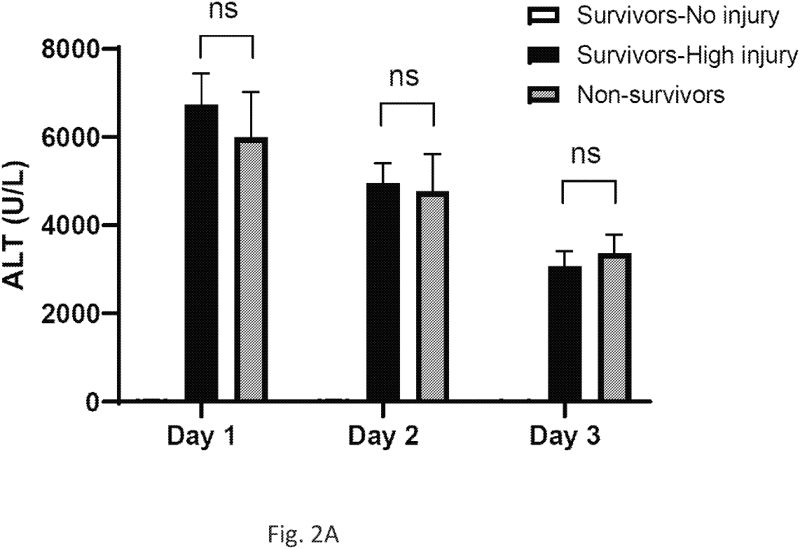

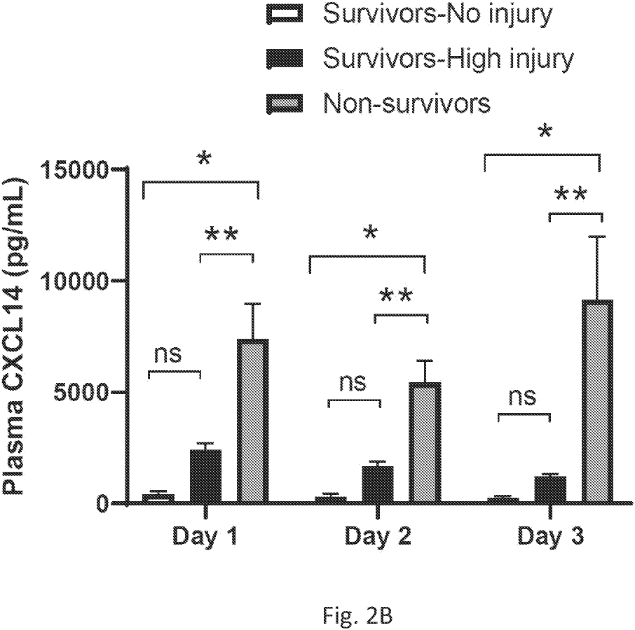

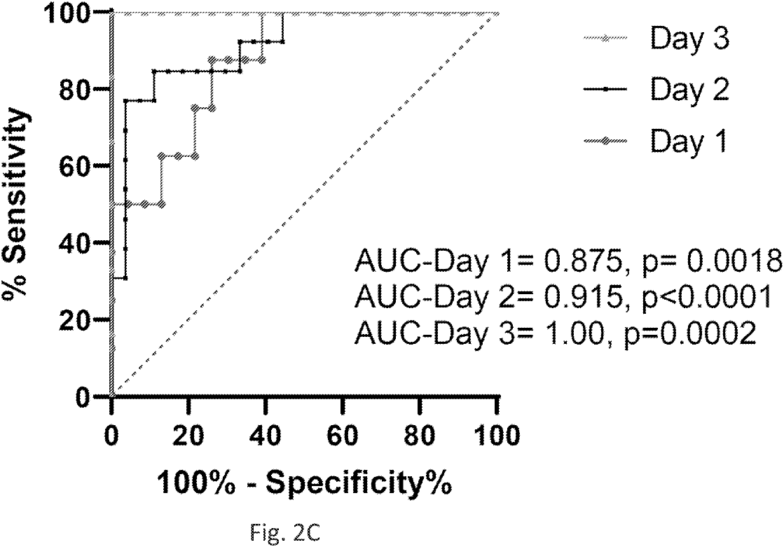

FIG. 2A shows liver injury as depicted by ALT levels over time in the three groups as indicated. Since the time from acetaminophen exposure until the patient going to the clinic for treatment is highly variable, the day of peak ALT as day 1 was set for all patients to streamline data. As seen in the FIG. 2A, ALT levels do not differ significantly between the survivors with high injury and the non-survivors. FIG. 2B shows plasma levels of CXCL14 in these patients, and no CXCL14 increase was evident patients with low injury.

However, the CXCL14 level in FIG. 2B was significant elevated in surviving patients with high injury at day 1, but it gradually decreased by day 3. In contrast, it was found that CXCL14 levels were much more elevated in non-survivors at day 1, and these levels were sustained at high levels by day 3 compared to the survivors. Again, the steep slope with no downward arc shows the need for a liver transplant, where low levels and downward arc shows the need to avoid a liver transplant.

To evaluate the use of plasma CXCL14 levels as a biomarker to predict patient outcome, an ROC analysis (FIG. 2C) was performed, which demonstrated that CXCL14 levels at all three days were predictive of death with 100% sensitivity at day 3. Thus, these data strongly indicate a significant difference of CXCL14 in plasma between potential survivors that do not need a liver transplant and potential non-survivors that need a transplant, when measured after acetaminophen overdose, which can be clinically used as a biomarker to predict patient outcome.

FIG. 2A includes a graph that shows the ALT levels in plasma. FIG. 2B includes a graph that shows the CXCL14 levels in plasma as measured by ELISA. FIG. 2C includes an receiver operatic characteristic (ROC) curve depicting prognostic relevance of CXCL14 data to predict outcome of patients after an acetaminophen exposure (overdose).

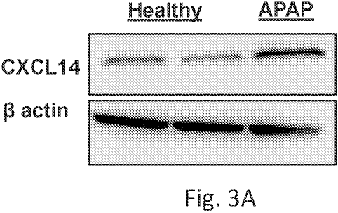

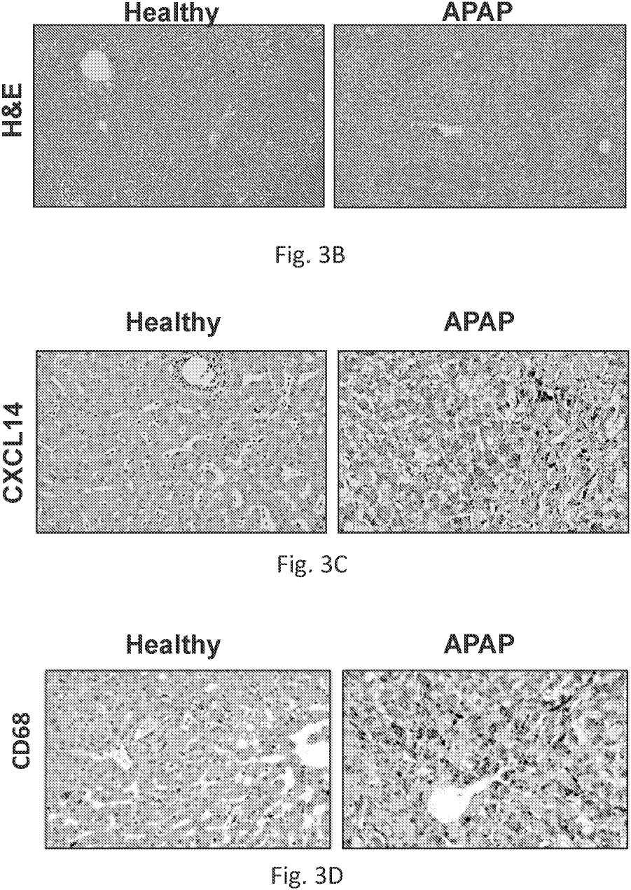

A western blot analysis on the removed liver from a transplant recipient after acetaminophen overdose was carried out to determine whether circulating CXCL14 levels also reflected levels within the human liver. Elevation in hepatic levels of CXCL14 are evident (FIG. 3A) indicating that the increased circulating CXCL14 levels are being produced in the liver. Therefore, the biological sample can be a blood sample (or plasma) or a liver sample. This is further confirmed by immunostaining of human liver sections, which show significant areas of necrosis by H&E staining as expected (FIG. 3B). Staining for CXCL14 within these sections indicated significant elevations confirming the western blot data (FIG. 3C). Since CXCL14 is a chemokine and the studies in the mouse model indicates that it could be involved in immune cell regulation, liver sections from this acetaminophen overdose patient were also stained for CD68 to evaluate hepatic macrophage infiltration. A significant increase in macrophage staining with CD68 was seen in the liver from acetaminophen overdose patient (FIG. 3D) confirming that changes seen in the mouse model are replicated in humans. Table 2 shows that the human sample also had an elevated level of CXCL14 at day 3 (6128 pg/mL) in serum with mild ALT level.

TABLE 2

| | Healthy | APAP overdose patient (day 3) |
|---|---|---|
| ALT (U/L) | 45.6 | 1620.9 |
| Serum CXCL14 (pg/mL) | 22.4 | 6128.12 |

We measured CXCL14 in plasma of acetaminophen-induced injury patients for three days. To make the data more reliable due to the complicated admission day and time of acetaminophen being taken, Day 1 is chosen as the day of peak ALT. We observed no CXCL14 increase in low injury patients (<100 pg/mL). However, this level is significant elevated in the high injury patients at day 1 (2500 pg/mL, p<0.05), but its level is gradually decreased by day 3 (900 pg/mL ±) as shown in FIG. 2A. In contrast, we found CXCL14 level is significant increased at day 1 (7000 pg/mL), but its level keep elevated by day 3 (8000 pg/mL) with a much higher level compared to the survivors (FIG. 2B). Next, we want to determine whether CXCL14 level in plasma can predict patient outcome. ROC analysis demonstrated CXCL14 level at all three days were predictive of death with 100% sensitivity at day 3 (FIG. 2C). Thus, these data strongly suggest a significant difference of CXCL14 in plasma between the survivors and non-survivors, which can be clinically used as a biomarker to predict patient outcome.

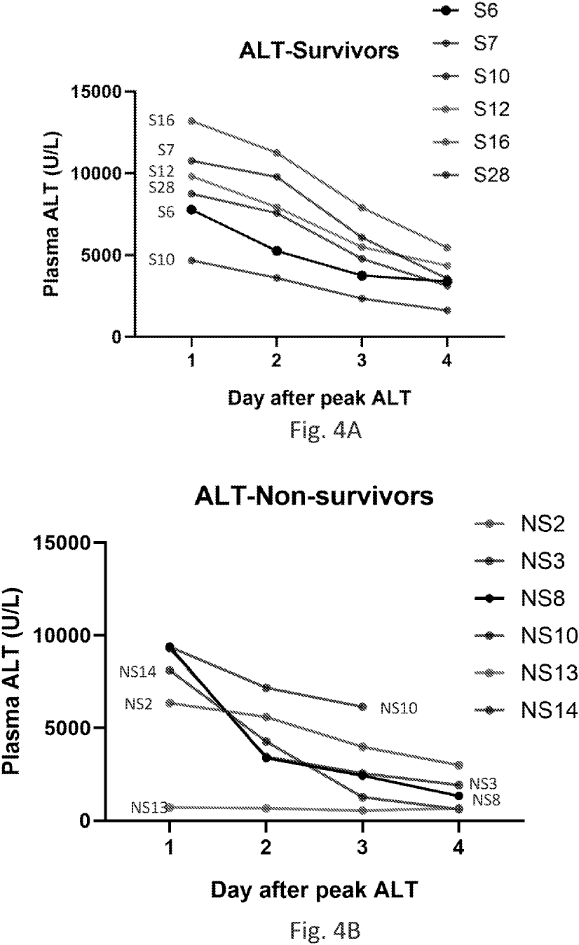

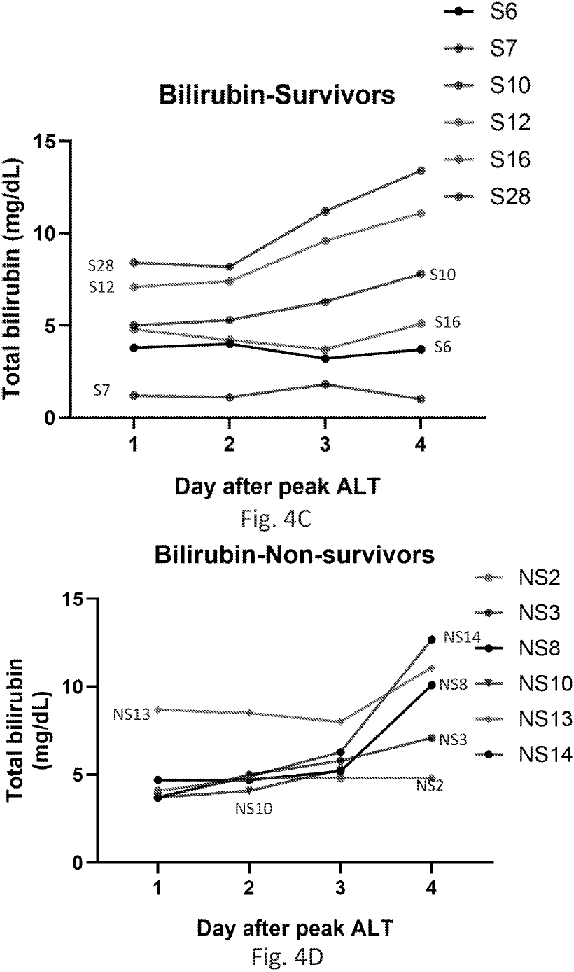

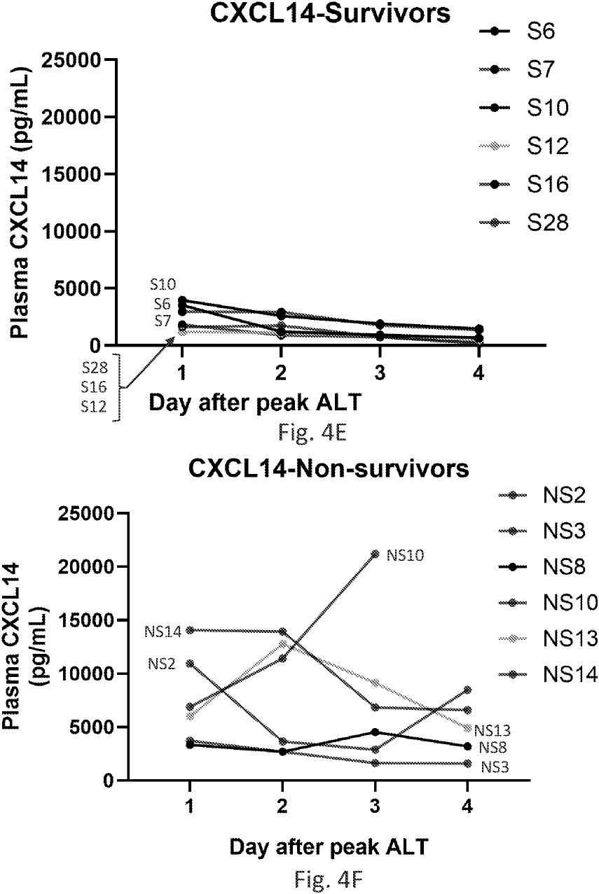

FIG. 4A includes a graph that shows the plasma ALT (U/L) versus the day after peak ALT for survivors S6, S7, S10, S12, S16 and S28. FIG. 4B shows the ALT (U/L) versus the day after peak ALT for non-survivors NS2, NS3, NS8, NS10, NS13, and NS14.

FIG. 4C includes a graph that shows the plasma total bilirubin (mg/dL) versus the day after peak ALT for survivors S6, S7, S10, S12, S16 and S28. FIG. 4D shows the plasma total bilirubin (mg/dL) versus the day after peak ALT for non-survivors NS2, NS3, NS8, NS10, NS13, and NS14.

FIG. 4E includes a graph that shows the plasma CXCL14 (pg/mL) versus the day after peak ALT for survivors S6, S7, S10, S12, S16 and S28. FIG. 4D shows the plasma CXCL14 (pg/mL) versus the day after peak ALT for non-survivors NS2, NS3, NS8, NS10, NS13, and NS14.

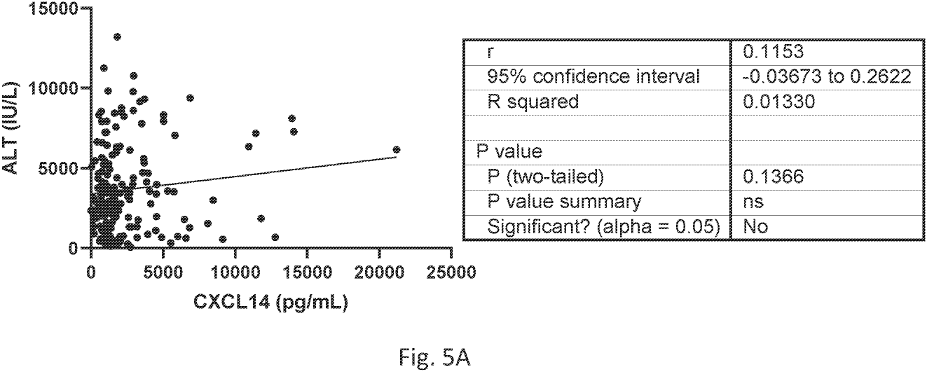

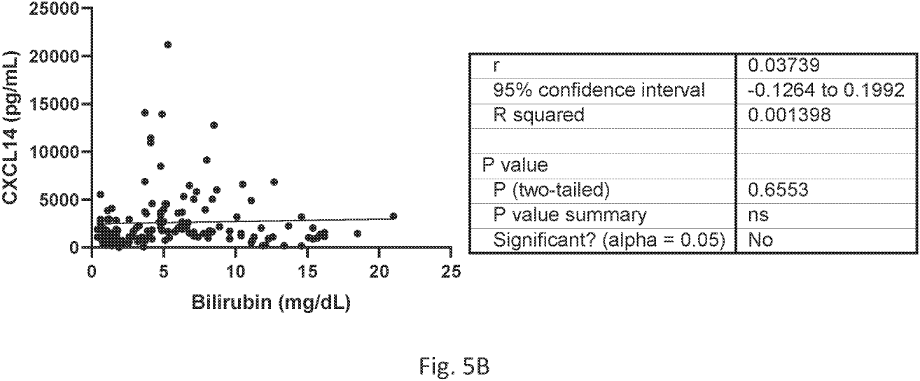

According to the data, plasma CXCL14 in the survival patients is high at first, but the level gradually decreased overtime. For example, patient S28 had a significant increase in bilirubin over time (12.5 mg/dL at day 4) which equal to a non-survival patient NS14 at day 4. However, patient S28 had high CXCL14 at day 1 (2111 pg/mL), but it was decreased significantly at day 4 (182 pg/mL). In contrast, NS14 started with very high CXCL14 level at day 1 (14000 pg/mL) and its level was still high at day 4 (6586 pg/mL). Moreover, correlation analysis between CXCL14 vs ALT or bilirubin showed no dependent relationship, as shown in FIGS. 5A-5B. Taken together, the data indicates CXCL14 is a better diagnosis marker to predict the outcome of acetaminophen overdose patients.

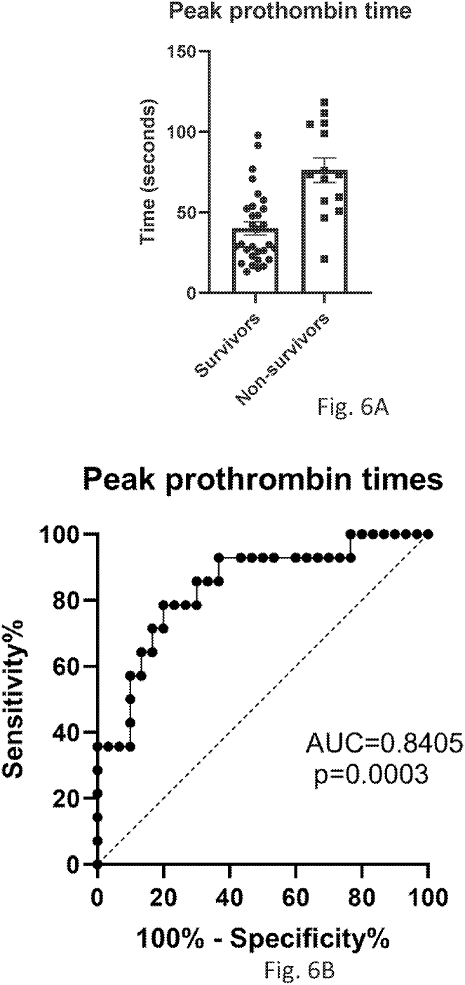

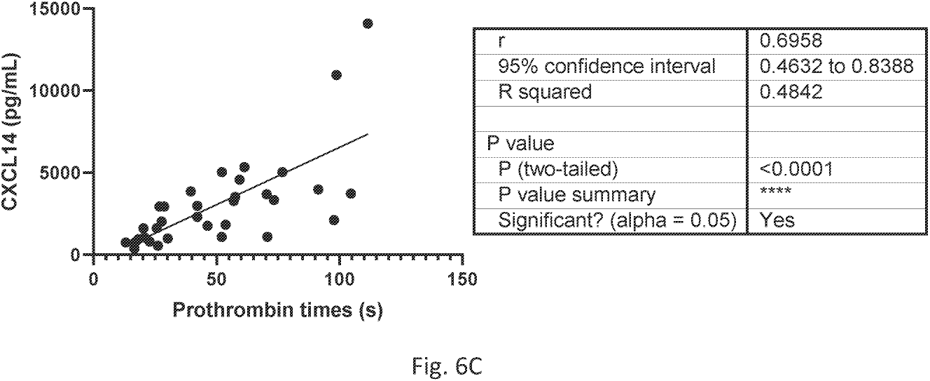

Additionally, prothrombin times were studied as an indicator of acetaminophen-induced liver failure. The prothrombin times were significantly higher in the non-survival patients (FIG. 5A). ROC analysis indicated significant differences in peak prothrombin times in survival and non-survival patients (AUC=0.84, p=0.0003) (FIG. 6A). In addition, a comparison was made of the correlation of CXCL14 at day 1 and peak thrombin times, and it was observed that there is a strong correlation (p<0.0001). Thus, CXCL14 and prothrombin times could be used together to predict patient outcomes early. The data strongly suggested that CXCL14 alone could be used as a predictive marker at later time (day 3 from peak injury). But, CXCL14 and prothrombin time together may give a better diagnosis at earlier time (day 1). Thus, high CXCL14 over 3,000 pg/mL plasma with peak prothrombin at day 1 can indicate a need for a liver transplant. The 1 day can be after peak ALT.

### Methods

Histology and Immunohistochemistry Formalin-fixed human and mouse tissue samples were embedded in paraffin and 5 µm sections were cut. Sections were stained with hematoxylin and eosin (H&E) for evaluation of the areas of necrosis. Additional liver sections were hydrated and went through antigen retrieval by boiling in sodium citrate buffer (pH 6.0). Liver sections were blocked in 3% BSA and serum then incubated with the primary antibodies overnight. Primary antibodies included anti-rabbit CD68 (Cell Signaling Technology, cat # 26042). Liver sections were washed and incubated in 3% hydrogen peroxide to block endogenous peroxidases. Biotinylated secondary antibody and DAB substrate were used to visualize the signals. Immunofluorescence staining was performed using 5 µm cryosections prepared from OCT-embedded tissue. Sections were blocked with 3% BSA and stained with anti-rabbit CXCL14 (Abcam, cat #264467). Fluorescence conjugated secondary antibodies Alexa Fluor 488 (Invitrogen, cat #A11034) goat anti rabbit was used.

Western blotting snap frozen tissue was homogenized in a CHAPs containing protein buffer and total protein was measured using the BCA assay (Pierce Scientific, Waltham, Massachusetts). Gel electrophoresis was carried out on protein lysates from individual samples, which were then transferred to a nitrocellulose blot.

ALT assay plasma alanine aminotransferase (ALT) activities were measured using an ALT test kit (Point Scientific, Inc, Canton, MI) per the manufacturer’s instruction.

All results were expressed as mean ± SE. Comparisons between multiple groups were performed with one-way ANOVA or, where appropriate, by two-way ANOVA, followed by a post hoc Bonferroni test. If the data were not normally distributed, we used the Kruskal-Wallis Test (nonparametric ANOVA) followed by Dunn’s Multiple Comparisons Test. P < 0.05 was considered significant. Receiver operating characteristic analysis curve was performed to determine optimal threshold values to discriminate between survivors and non-survivors.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

Blackmore L, Bernal W. Acute liver failure. Clin Med (Lond). 2015 Oct; 15(5): 468-72.

Akakpo JY, Jaeschke MW, Ramachandran A, Curry SC, Rumack BH, Jaeschke H. Delayed administration of N-acetylcysteine blunts recovery after an acetaminophen overdose unlike 4-methylpyrazole. Arch Toxicol. 2021 Oct; 95 (10) : 3377-3391.

Bernal W, Wendon J, Rela M, Heaton N, Williams R. Use and outcome of liver transplantation in acetaminophen-induced acute liver failure. Hepatology. 1998 Apr; 27 (4): 1050-5.

1. A method of determining a likelihood of liver transplant in a subject, comprising:
providing a subject having consumed acetaminophen and having been diagnosed with hepatotoxicity;
obtaining a plurality of blood samples from the subject at a series of time points over a plurality of days;
measuring an amount of CXCL14 in the blood sample at the series of time points;
comparing the amount of CXCL14 in the blood samples to the series of time points to determine a trend in the amount of the CXCL14 in the blood samples over a time period beginning at an time point and continuing through the series of time points;
identifying a trend of:
(a) the amount of CXCL14 increases over the time period;
(b) the amount of CXCL14 increases and then becomes substantially constant; or
(c) the amount of CXCL14 has a maximum and then decreases;
selecting the trend in the amount of CXCL14 in the blood over the time period that matches the trend of (a), (b), or (c);
determining a treatment of the subject based on the trend in the amount of CXCL14 in the blood samples over the time period, wherein the treatment is selected from:
when the amount of CXCL14 increases over the time period, the treatment is determined to be a liver transplant;
when the amount of CXCL14 increases and then becomes substantially constant, the treatment is determined to be a liver transplant; or
when the amount of CXCL14 has a maximum and then decreases, the treatment is determined to exclude a liver transplant;
preparing a report that identifies the treatment for the subject; and
providing the report to the subject or medical professional or medical computing system.

2. The method of claim 1, wherein the time period is at least 2 days.

3. The method of claim 1, wherein the time period is at least 3 days.

4. The method of claim 1, wherein in trend (c) the amount of CXCL14 starts at a maximum and then decreases.

5. The method of claim 1, wherein in trend (c) the amount of CXCL14 increases to a maximum and then decreases.

6. The method of claim 1, wherein when the amount of CXCL14 is at least 3,000 pg/mL in plasma from the blood, the treatment is the liver transplant.

7. The method of claim 1, wherein when the amount of CXCL14 is at least 5,000 pg/mL in plasma, the treatment is the liver transplant.

8. The method of claim 1, further comprising measuring at least one of ALT, bilirubin, or prothrombin.

9. The method of claim 8, wherein data from the measuring of at least one of ALT, bilirubin, or prothrombin supports the treatment.

10. The method of claim 9, wherein measurement of prothrombin at a plurality of time points supports the treatment.

11. A method of treating a subject having hepatotoxicity, comprising:
providing a subject having consumed acetaminophen and having been diagnosed with hepatotoxicity;
obtaining a plurality of blood samples from the subject at a series of time points over a plurality of days;
measuring an amount of CXCL14 in the blood sample at the series of time points;
comparing the amount of CXCL14 in the blood samples to the series of time points to determine a trend in the amount of the CXCL14 in the blood samples over a time period beginning at the initial time point and continuing through the series of time points;
identifying a trend of:
(a) the amount of CXCL14 increases over the time period;
(b) the amount of CXCL14 increases and then becomes substantially constant; or
(c) the amount of CXCL14 has a maximum and then decreases;
selecting the trend in the amount of CXCL14 in the blood over the time period that matches the trend of (a), (b), or (c);
determining a treatment of the subject based on the trend in the amount of CXCL14 in the blood samples over the time period, wherein the treatment is selected from:
when the amount of CXCL14 increases over the time period, the treatment is determined to be a liver transplant;
when the amount of CXCL14 increases and then becomes substantially constant, the treatment is determined to be a liver transplant; or
when the amount of CXCL14 increases to a maximum and then decreases, the treatment is determined to exclude a liver transplant; and coordinating performance of the treatment on the subject.

12. The method of claim 11, further comprising performing the treatment on the subject.

13. The method of claim 11, wherein the treatment includes the liver transplant.

14. The method of claim 11, wherein the treatment excludes the liver transplant.

15. The method of claim 11, wherein the time period is at least 2 days.

16. The method of claim 11, wherein the time period is at least 3 days.

17. The method of claim 11, wherein in trend (c) the amount of CXCL14 starts at a maximum and then decreases.

18. The method of claim 11, wherein in trend (c) the amount of CXCL14 increases to a maximum and then decreases.

19. The method of claim 11, wherein when the amount of CXCL14 is at least 3,000 pg/mL in plasma from the blood, the treatment is the liver transplant.

20. The method of claim 1, wherein when the amount of CXCL14 is at least 5,000 pg/mL in plasma, the treatment is the liver transplant.
